(19) United States
(12) Patent Application Publication
(10) Pub. No.: US 2022/0115121 A1
(43) Pub. Date: Apr. 14, 2022

(54) MEASURING DEVICE AND MEASURING SYSTEM

(71) Applicant: ISHIDA CO., LTD., Kyoto-shi, Kyoto (JP)

(72) Inventors: Taku TERAMOTO, Kyoto-shi, Kyoto (JP); Yoshito KUNISAKI, Kyoto-shi, Kyoto (JP); Makoto NAKATANI, Kyoto-shi, Kyoto (JP)

(21) Appl. No.: 17/275,546

(22) PCT Filed: Sep. 2, 2019
(86) PCT No.: PCT/JP2019/034483

§ 371 Date: Mar. 11, 2021

(30) Foreign Application Priority Data
Sep. 14, 2018 (JP) 2018-172947

(51) Int. Cl. G16H 40/63; A61B 5/20; A61B 5/00; G06K 7/14; G16H 10/60; G01G 19/18

(52) U.S. Cl. CPC G16H 40/63; A61B 5/208; A61B 5/742; A61B 5/7278; G01G 19/18; A61B 5/7282; A61B 5/0004; G06K 7/1413; G16H 10/60; A61B 5/002

(57) ABSTRACT

A measuring device includes a weighing unit configured to measure a mass value of a urine bag, a calculation unit configured to calculate at least one of a voided volume and a urinary output per unit time on the basis of the mass value weighed by the weighing unit, a touch panel display configured to display urinary output information related to at least one of the voided volume and the urinary output per unit time calculated by the calculation unit, and an output unit configured to generate a code including the urinary output information, output the code to the touch panel display, transmit the urinary output information by wireless communication, and/or print the urinary output information or the code.

### TECHNICAL FIELD

The present invention relates to a measuring device and a measuring system.

### BACKGROUND ART

In hospitals and the like, urine bags are used for patients having. difficulty in urinating by themselves after surgery with general anesthesia, or the like. A urine bag stores urine discharged from a patient (for example, refer to Patent Literature 1).

### CITATION LIST

### Patent Literature

[Patent Literature 1] Japanese Unexamined Utility Model Publication No. H06-003314

### SUMMARY OF INVENTION

### Technical Problem

Generally, the amount of urine accumulated in a urine bag is regularly and visually checked by a staff member of a hospital, such as a nurse. The staff member checks the amount of urine on the basis of a scale marked on the urine hag, discharges the urine from the urine bag to a graduated cylinder, and weighs the urine, thereby recording the amount of urine in a management sheet or the like. Accordingly, for example, the physical condition or the like of a patient after surgery is managed, or timings of taking urine from a urine bag are adjusted or the like. In recent years, patient information has come to be managed using electronic medical records. For this reason, management methods in the related art require the work of registering a urinary output recorded in a management sheet in electronic medical records. In this case, when a urinary output is registered in electronic medical records, a human error may occur, or when there is a need to cope with a plurality of patients, a burden may be placed on a staff member.

An object of an aspect of the present invention is to provide a measuring device and a measuring system capable of achieving work efficiency.

### SOLUTION TO PROBLEM

According to an aspect of the present invention, there is provided a measuring device acquiring information related to urine stored in a urine hag. The measuring device includes a weighing unit configured to measure a mass value of the urine bag; a calculation unit configured to calculate at least one of a voided volume and a urinary output per unit time on the basis of the mass value weighed by the weighing unit; a display unit configured to display urinary output: information related to at least one of the voided volume and the urinary output per unit time calculated by the calculation unit; and an output unit configured to generate a code including the urinary output information, output the code to the display unit, transmit the urinary output information by wireless communication, and/or print the urinary output information or the code.

In the measuring device according to the aspect: of the present invention, the output unit generates a code including urinary output information, outputs the code to the display unit, transmits the urinary output information by wireless communication, and/or prints the urinary output information or the code. In this configuration, urinary output. information can be automatically acquired using a different terminal by reading a code generated by the output unit and displayed in the display unit using a different terminal. In addition, urinary output information can be automatically acquired using a different terminal by receiving the urinary output information transmitted by the output unit by wireless communication using a different terminal. In addition, a sheet or the like in which urinary output information or a code is printed can be acquired. In this manner, in the measuring device, work of a staff member inputting a urinary output to electronic medical records for each patient can be omitted. Therefore, in the measuring device, it is possible to avoid occurrence of a human error when a urinary output is registered in electronic medical records or a burden on a staff member when there is a need to cope with a plurality of patients. As a result, in the measuring device, work efficiency can be achieved.

In the measuring device, since not only urinary output information can be displayed in the display unit but also urinary output information is output in a code or by wireless communication, a staff member can acquire urinary output information without touching the urine bag. Therefore, a hygienic aspect when acquiring urinary output information can be ensured. Accordingly, at the time of rounds or the like to a hospital room by a staff member, a computer or the like can be carried into the hospital room, and urinary output information can be registered in electronic medical records. Therefore, since urinary output information can be registered in electronic medical records immediately after the urinary output information is acquired, work efficiency can be achieved.

According to the embodiment, the output unit may transmit the urinary output information by short-distance wireless communication.

In this configuration, urinary output information can he acquired in a non-contact manner by bringing a reader provided in (connected to) a different terminal near the measuring device. Therefore, urinary output information (target) of the measuring device can he reliably acquired.

According to the embodiment, the measuring device may further include an acquisition unit configured to acquire target person information indicating a target person using the urine bag, and a management unit configured to manage the target person information acquired by the acquisition unit and the urinary output information in association with each other. In this configuration, urinary output information can be managed for each target person (patient or the like).

According to the embodiment:, a reading device reading a code indicating the target person information may be able to be connected to the measuring device. The acquisition unit may acquire the target person information on the basis of the code read by the reading device, In this configuration, target person information and urinary output information can he accurately associated with each other. Therefore, it is possible to reliably avoid occurrence of a problem such as occurrence of an error in association between target person information and urinary output information.

According to the embodiment, the measuring device may further include a determination unit configured to determine anuria, oliguria, and polyuria on the basis of the urinary output information and a. threshold set in advance. The output unit may output notification information when the determination unit determines anuria, oliguria, or polyuria. In this configuration, a staff member can be notified of anuria, oliguria, or polyuria.

According to another aspect of the present invention, there is provided a measuring system including the measuring device described above, and a management device configured to acquire information related to urine acquired by the measuring device.

In the measuring system according to the aspect of the present invention, the output unit of the measuring device generates a code including, urinary output information, outputs the code to the display unit, transmits the urinary output information by wireless communication, and/or prints the urinary output information or the code. In this configuration, urinary output information can be automatically acquired using the management device by reading a code generated by the output unit and displayed in the display unit using an input unit of the management device. In addition, urinary output information can be automatically acquired using the management device by receiving the urinary output information transmitted by the output unit by wireless communication using the management device. In addition, a sheet or the like in which urinary output information or a. code is printed can be acquired. In this manner, in the measuring system, work of a staff member inputting a urinary output to electronic medical records for each patient can be omitted. Therefore, in the measuring system, it is possible to avoid occurrence of a human error when a urinary output is registered in electronic medical records or a burden on a staff member when there is a need to cope with a plurality of patients. As a result, in the measuring system, work efficiency can be achieved.

### Advantageous Effects of Invention

According to the aspects of the present invention, work efficiency can be achieved.

### BRIEF DESCRIPTION OF DRAWINGS

FIG. 1 is a view illustrating a measuring system according to a first embodiment.

FIG. 2 is a perspective view illustrating a measuring device.

FIG. 3 is another perspective view illustrating the measuring device.

FIG. 4 is a view illustrating a configuration of the measuring device of the measuring system illustrated in FIG I.

FIG. 5 is a view illustrating a configuration of a weighing unit.

FIG. 6 is a view illustrating an example of a screen displayed in a touch panel display.

FIG. 7 is a view illustrating another example of a screen displayed in the touch panel display.

FIG. 8 is a view illustrating another example of a screen displayed in the touch panel display.

FIG. 9 is a view illustrating another example of a screen displayed in the touch panel display.

FIG. 10 is a view illustrating another example of a screen displayed in the touch panel display.

FIG. 11 is a view illustrating another example of a screen displayed in the touch panel display.

FIG. 12 is a view illustrating another example of a screen displayed in the touch panel display.

FIG. 13 is a view illustrating a configuration of a management device.

FIG. 14 is a view illustrating an example of a screen displayed in a display unit.

FIG. 15 is a view illustrating another example of a screen displayed in the display unit.

FIG. 16 is a view illustrating a measuring system according to a second embodiment.

FIG. 17 is a view illustrating a configuration of the measuring device of the measuring system illustrated in FIG. 16.

FIG. 18 is a view illustrating another example of a screen displayed in the touch panel display.

FIG. 19 is a view illustrating a configuration of the management device of the measuring system illustrated in FIG. 16.

FIG. 20 is a perspective view illustrating a measuring device according to a different embodiment.

FIG. 21 is a perspective view illustrating the measuring device according to the different embodiment.

FIG. 22 is a front view of the measuring device according to the different embodiment.

FIG. 23 is a view illustrating another example of a screen displayed in the touch panel display of the measuring device according to the different embodiment.

### DESCRIPTION OF EMBODIMENTS

Hereinafter, preferable embodiments of the present invention will be described in detail with reference to the accompanying drawings. In description of the drawings, the same reference signs are applied to elements which are the same or corresponding, and duplicate description will he omitted.

[First Embodiment]

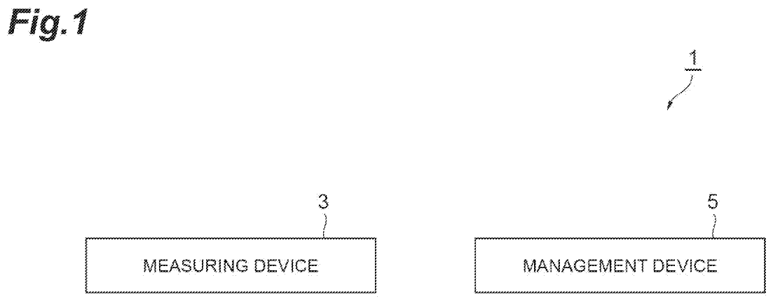

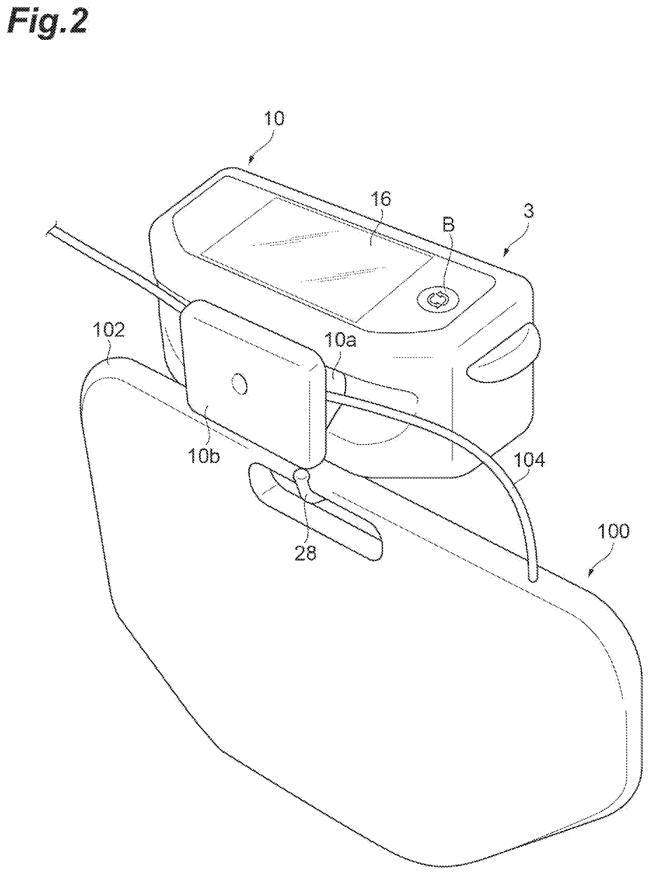

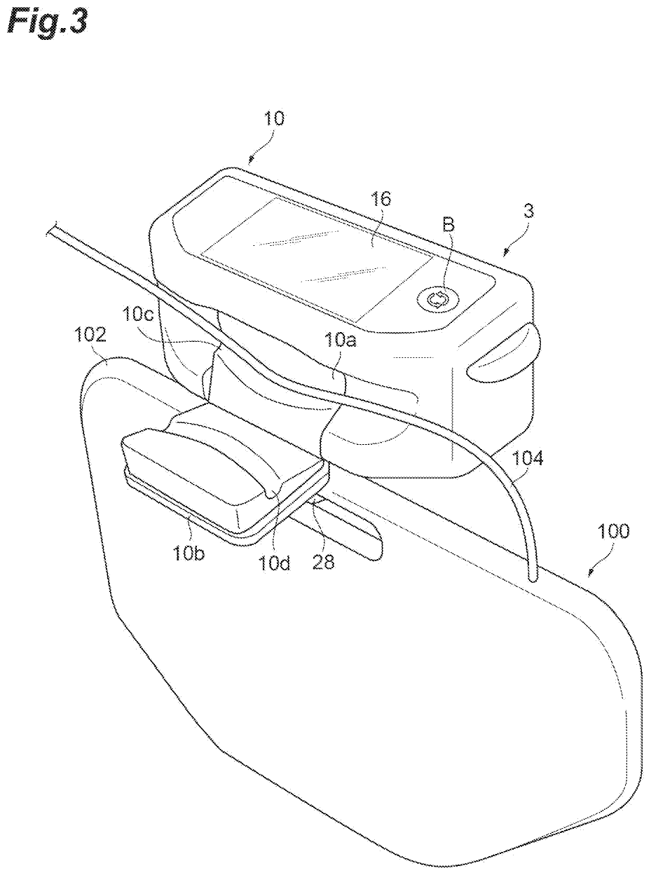

As illustrated in FIG. 1, a measuring system 1 includes a measuring device 3 and a management device 5. The measuring system 1 may include one or a plurality of measuring devices 3 and management devices 5. For example, the measuring system 1 manages patient's urine stored in a urine hag 100 (refer to FIGS. 2 and 3) in a hospital or the like.

The measuring device 3 acquires information related to urine stored in the urine bag 100. As illustrated in FIGS. 2 and 3, the urine bag 100 has a storage unit 102 storing urine, and a urine guide tube 104 for introducing urine into the storage unit 102. The urine bag 100 may be provided with a voidance tube for discharging urine stored in the storage unit 102. Various commercially available bags can be used as the urine bag 100.

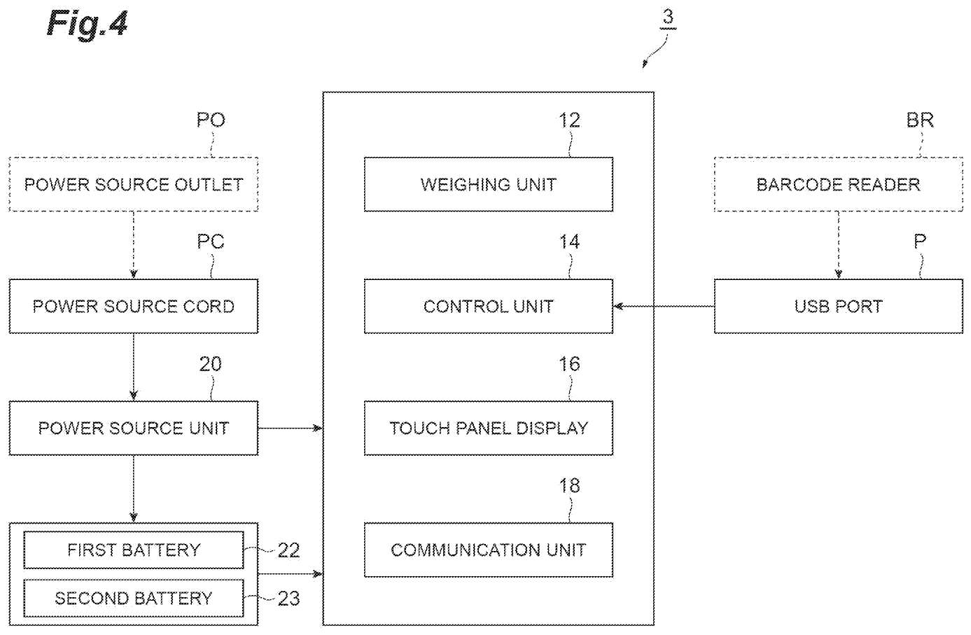

As illustrated in FIG. 4, the measuring device 3 has a housing 10 (refer to FIGS. 2 and 3), a weighing unit 12, a control unit (a calculation unit, an output unit, an acquisition unit, a management unit, and a determination unit) 14, a touch panel display (a display unit) 16, a communication unit 18, a power source unit 20, a first battery 22, and a. second battery 23.

For example, the housing 10 is formed of an ABS resin or the like. As illustrated in FIGS. 2 and 3, the housing 10 has a holding portion 10a and a fixing portion 10b. The holding portion 10a is provided with a groove (a recessed portion) 10c in which the urine guide tube 104 is disposed. The fixing portion 10b is provided at a central portion of the holding portion 10a. The fixing portion 10b is provided in a swingable manner in the holding portion 10a. The fixing portion 10b is provided with a groove 10d in which the urine guide tube 104 is disposed. As illustrated in FIG. 3, the urine guide tube 104 can be attached to the holding portion 10a, and the urine guide tube 104 can be detached from the holding portion 10a by opening, the fixing portion 10b. As illustrated in FIG. 2, the urine guide tube 104 can he sandwiched between the groove 10c and the groove 10d. and the urine guide tribe 104 can he fixed to the holding portion 10a. by closing the fixing portion 10b. The fixing portion 10b restricts movement of the urine guide tube 104 in the holding portion 10a.

As illustrated in FIG. 4, the housing 10 is provided with a measurement button B for starting measurement (refer to FIGS. 2 and 3) and a USB port 1. in addition, the housing 10 is provided with a connection portion (not illustrated) to which a power source cord PC for supplying power to the measuring device 3 is connected.

A terminal to be connected to a connection portion of the measuring device 3 is provided at one end of the power source cord PC, and an outlet plug to be connected to a power source outlet PO is provided at the other end of the power source cord PC. When the outlet plug of the power source cord PC is connected to the power source outlet PO, the measuring device 3 may receive supply of power from the power source outlet PO. Power supplied from the power source outlet. PO is supplied to the power source unit 20.

A barcode reader (a reading device) BR can he connected to the USB port P. The barcode reader BR reads barcodes including patient information. Patient information may include the name or the like of a. I0 patient. For example, a barcode is displayed in a band attached to a wrist of a patient. The barcode reader BR outputs read patient information to the control unit 14.

The housing 10 is provided with an attachment portion (not illustrated) for attaching the measuring device 3 to a bed, a drip infusion stand, or the like. For example, the attachment portion is disposed on a rear surface (a back surface) side of the housing 10. The attachment portion may be provided in a manner of being attachable/detachable with respect to the housing 10. The attachment portion can be switched in accordance with an attachment target (a bed, a drip infusion stand, or the like).

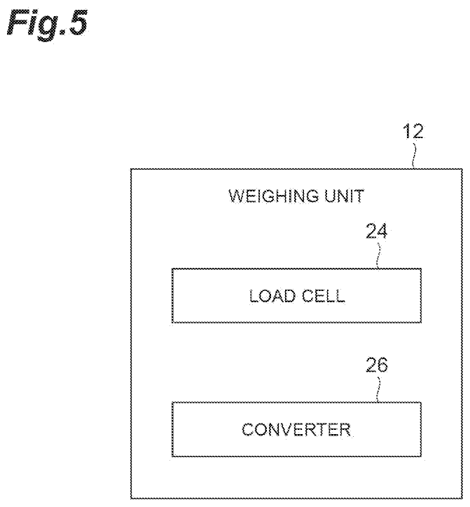

The weighing unit 12 measures the mass value of a urine bag. As illustrated in FIG. 5, the weighing unit 12 has a load cell 24 and a converter 26.

The load cell 24 is connected to the housing 10 and a hook 28. Specifically, in the load cell 24, one end thereof is fixed to the housing 10, and the hook 28 is provided at the other end thereof. For example, the hook 28 is formed of a resin. The hook 28 can move (swing) to a position where the urine bag 100 can be interlocked, that is, an interlock position (a position illustrated in FIG. 2) protruding from the housing 10, and an accommodation position of being accommodated inside the housing 10 when the urine bag 100 is not interlocked. The hook 28 may have any shape as long as the urine hag 100 can be held (the urine bag 100 does not fall off) with the shape.

The load cell 24 detects the mass of the storage unit 102 of the urine bag 100 interlocked with the hook 28. The load cell 24 detects I 0 the mass of the storage unit 102 of the urine bag 100 and outputs weighing data (a mass value) regarding the mass to the converter 26. Weighing data output by the load cell 24 is an analog signal such as a voltage corresponding to the mass of an article to be weighed. The load cell 24 is configured to include an element measuring a distortion of a semiconductor pressure sensor or the like. The load cell 24 is not particularly limited, and various kinds of known load. cells can be employed.

The converter 26 converts weighing data as an analog signal output from the load cell 24 into weighing data as a digital signal. The converter 26 outputs weighing data to the control unit

The control unit 14 is configured to have a central processing unit (CPU), a read only, memory (ROM), a random access memory (RAM), a hard disc drive (HDD), and the like. The control unit 14 receives an input from an operator and performs various kinds of setting of the measuring device 3. When a predetermined operation is performed in a standby screen D1 of the touch panel display 16, the control unit 14 causes the touch panel display 16 to display a setting screen.

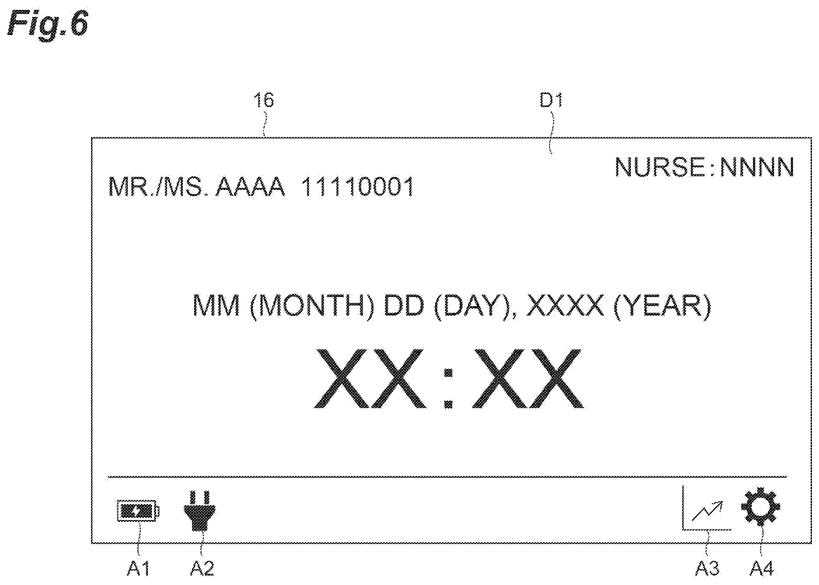

As illustrated in FIG. 6, the standby screen D1 displays “patient name”, “patient ID”, “nurse name”, “date and time”, “state icon”, “selection icon”, and the like. Regarding the “state icon”, for example, a residual quantity icon A1 indicating a state (residual quantity) of the first battery 22 and an icon A2 indicating a connection state of the power source cord PC are displayed. Regarding the “selection icon”, a history icon A3 and a setting icon A4 are displayed. When the setting icon A4 is touched in the standby screen D1 displayed in the touch panel display 16, the control unit 14 causes the touch panel display 16 to display the setting screen.

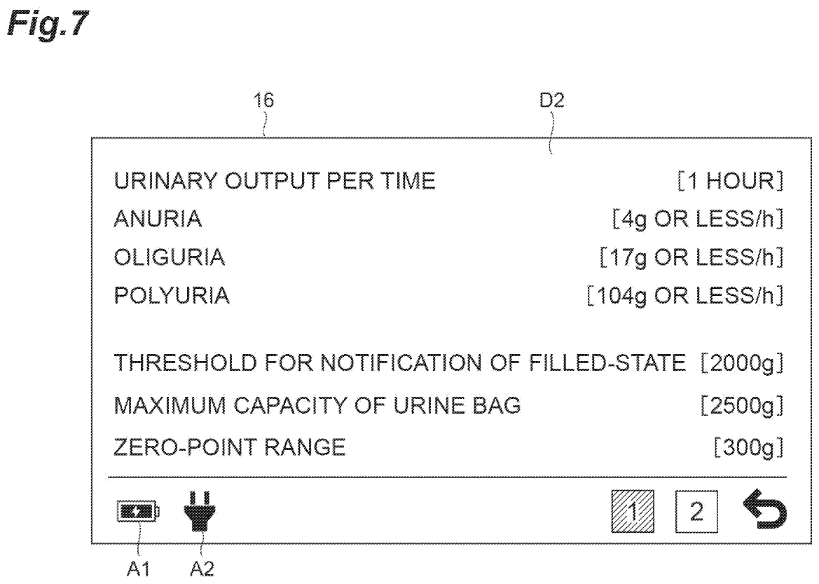

As illustrated in FIG. 7, for example, parameters can be set in a setting screen D2. For example, regarding parameter setting items, the setting screen D2 displays “urinary output per time”, “anuria”, “oliguria”, “polyuria”, “threshold for notification of filled-state”, “maximum capacity of urine bag”, “zero-point range”, and the like. For example, regarding the “urinary output per time”, “1 minute (g/min)”, “1 hour (g/hour)”, or “1 day (g/day)” can be set. Numerical values (thresholds) can be set for “anuria”, “oliguria”, “polyuria”, “threshold for a notification of filled-state”, “maximum capacity of urine bag”, and “zero-point range”. Numerical values for “anuria”, “oliguria”, and. “polyuria” may be automatically calculated and displayed. by inputting the body weight of a patient.

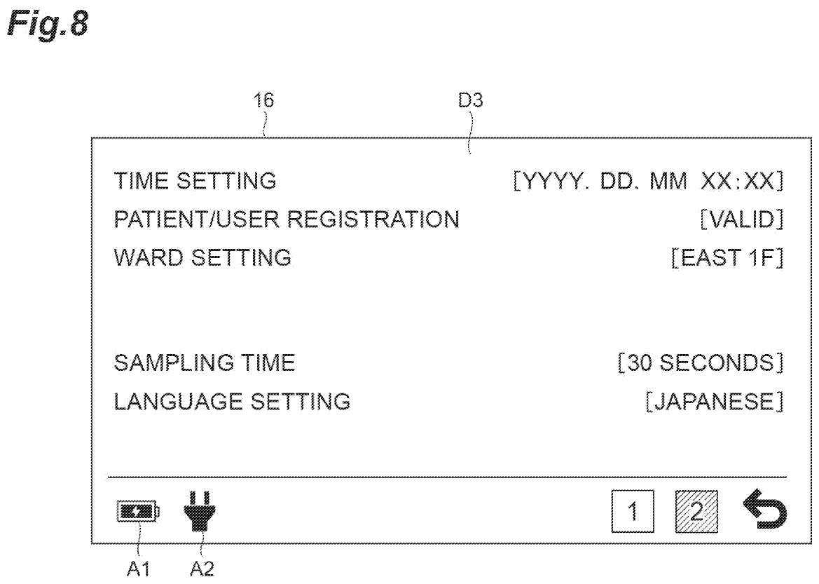

As illustrated in FIG. 8, for example, various kinds of setting can be performed in a setting screen D3. In the setting screen D2, “time settine”, “patient/user information”, “ward setting”, “sampling time”, and “language setting” can be set. Regarding the “language setting”, for example, Japanese, English, or the like can be selected.

The control unit 14 inputs patient information output from the barcode reader BR and registers the patient information. When a predetermined operation is performed by an operator for the item “patient/user registration” in the setting screen 12, the control unit 14 causes the barcode reader BR to start reading. When patient information output from the barcode reader BR is received, the control unit 14 manages the patient information and urinary output information (which will be described below) in association with each other.

The control unit 14 calculates a voided volume and a urinary output per unit time on the basis of the mass value weighed by the weighing unit 12. The control unit 14 calculates a weight from weighing data output from the converter 26 and calculates the weight (voided volume) [g] of urine by subtracting a tare weight (a weight including the urine bag 100 and the like) from the weight. A control unit 54 stores urinary output information indicating the calculated weight of urine in the HDD or the like. The control unit 14 calculates a urinary output [g] per unit time on the basis of the calculated weight of urine, The control unit 14 calculates a urinary output per unit time (1 hour or 1 day) in accordance with contents of setting. The control unit 14 stores the calculated urinary output per unit time in the HDD or the like.

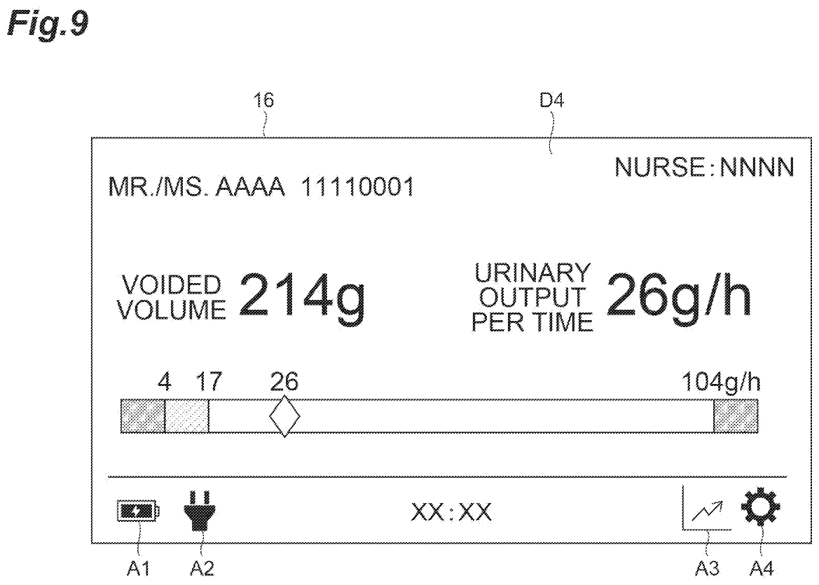

The control unit 14 causes the touch panel display 16 to display the voided volume and the urinary output per unit time. As illustrated in FIG. 9, the control unit 14 causes a measurement screen D4 to display a numerical value [g] for “voided volume” and a numerical value [g] for “urinary output per time”. The control unit 14 causes the measurement screen D4 to visually display current urinary output per unit times for anuria, oliguria, and polyuria. Specifically, the control unit 14 causes a bar in which parameters for anuria, oliguria, and polyuria are displayed to he displayed and causes a urinary output per unit time to be displayed on the bar.

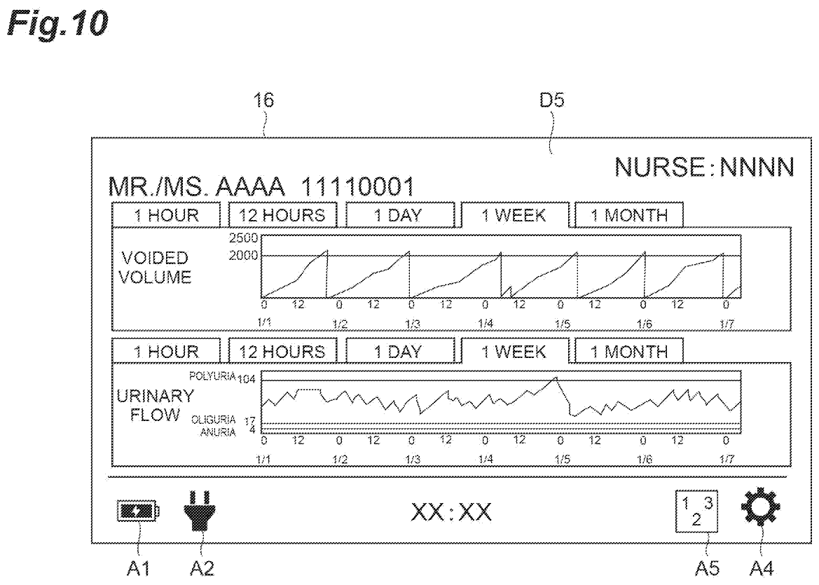

The control unit 14 causes the touch panel display 16 to display history of urinary outputs. When the history icon A3 is touched in the standby screen DI. the control unit 14 causes history of the amount of urine to be displayed. As illustrated in FIG. 10, in a history screen D5, history regarding “voided volume” and “urinary flow (urinary output per unit time)” is displayed in a graph. For example, the history can he displayed in units of “1 hour”, “12 hours”, “1 day”, “1 week”, and “1 month”. In the example illustrated in FIG. 10, history of 1 week is illustrated. When a measurement icon A5 is touched, the control unit. 14 causes the touch panel display 16 to display the measurement screen D4 illustrated in FIG. 9.

The control unit 14 determines anuria, oliguria, or polyuria on the basis of a urinary output per unit time. The control unit 14 determines anuria, oliguria, or polyuria on the basis of parameters set regarding anuria, oliguria, and polyuria set in advance. When the control unit 14 determines anuria, oliguria, or polyuria, the control unit. 14 outputs notification information for causing the touch panel display 16 to display anuria, oliguria, or polyuria to the touch panel display 16.

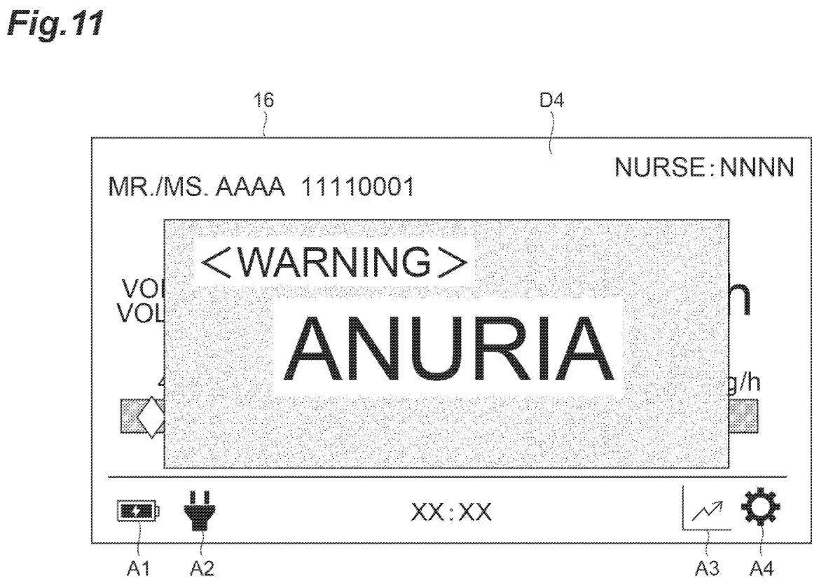

As illustrated in FIG. 11, for example, the control unit 14 causes the touch panel display 16 to display a warning pop-up. The control unit 14 may issue a notification using a buzzer or the like m addition to display in the touch panel display 16. When the voided volume becomes equal to or greater than the threshold for a notification of filled-state, the control unit 14 may output notification information for notifying that the urine bag 100 is in a :filled-state to the touch panel display 16 on the basis of urinary output information.

The control unit 14 acquires the state of the first battery 22. The control unit 14 acquires the residual quantity of the first battery 22.

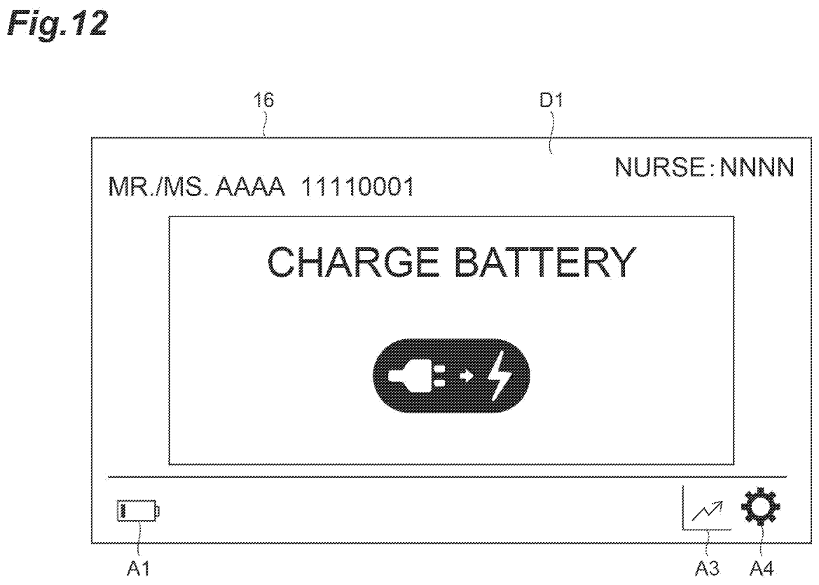

The control unit 14 controls display of the residual quantity icon A1 displayed in the touch panel display 16 in accordance with the residual quantity of the first battery 22. When the residual quantity of the first battery 22 becomes equal to or smaller than the threshold, the control unit 14 causes the touch panel display 16 to display that the residual quantity has decreased. As illustrated in FIG. 12, the control unit 14 causes the touch panel display 16 to display a pop-up indicating that. residual quantity of the first battery 22 has decreased (prompting a user to perform charging). The control unit 14 may issue a notification using a buzzer or the like in addition to display in the touch panel display 16.

When a predetermined operation is performed in the touch panel display 16, the control unit 14 outputs urinary output information to the communication unit 18. When an operation of instructing transmission of urinary output information is performed in the touch panel display 16, the control unit 14 outputs stored urinary output information to the communication unit 18. The control unit 14 causes the communication unit 18 to transmit the urinary output information for a predetermined period of time.

The touch panel display 16 can display texts, images, or the like and receives an input of an operator. In the present embodiment, the touch panel display 16 can perform display in colors (256 or more colors). Contents of display in the touch panel display 16 can be controlled by the control unit 14.

The communication unit 18 is a wireless module for performing short-distance wireless communication. For example, the communication unit 18 performs communication in conformity with near field communication (NEC). The communication unit 18 transmits urinary output information output from the control unit 14. For example, the communication unit 18 is disposed in the vicinity of an upper surface part of the housing 10. Accordingly, when a reader R (which will be described below) of the management device 5 is brought near the upper surface part of the housing 10, the reader R can receive urinary output information transmitted from the communication unit 18.

The power source unit 20 supplies power to each unit of the measuring device 3. Specifically, the power source unit 20 supplies power to the weighing unit 12, the control unit 14, the touch panel display 16, and the first battery 22. The power source unit 20 receives supply of power from the power source outlet PO via the power source cord PC.

When power is not supplied from the power source unit 20, the first battery 22 supplies power to the weighing unit 12, the control unit 14, and the touch panel display 16. For example, the first battery 2.2 is a lithium-ion battery. The first battery 22 is charged upon reception of supply of power from the power source unit 20. The first battery 22 is charged by a charging device (not illustrated) connected to the power source unit 20. When power is no longer supplied from the power source unit 20, the first battery 22 starts supply of power. The first battery 22 may be attachable/detachable with respect to the housing 10. In tins case, the first battery 22 can be charged by a charger provided in a nurse station or the like.

The second battery 23 is a power source for memory back-up. For example, the second battery 23 is a lithium-ion battery. The second battery 23 may have a smaller capacity than the first battery 22. When power is not supplied. from the power source unit 20 and the first battery 22 is detached from the housing 10 or when the residual quantity of the first battery 22 has decreased, the second battery 23 supplies power to at least the control unit 14. The second battery 23 is charged upon reception of supply of power from the power source unit 20. in the measuring device 3, even when power is not supplied from the power source unit 20 and the first battery 22, setting and the like in the measuring device 3, such as setting of a timepiece, measurement results, and measurement history can be maintained by the second battery 23.

The management device 5 functions as a host device integrally managing the measuring device 3. The management device 5 manages data measured by the measuring device 3. For example, the management device 5 may be constituted of a dedicated computer, or a commercially available personal computer in which software (a program for electronic medical records) is installed may be used.

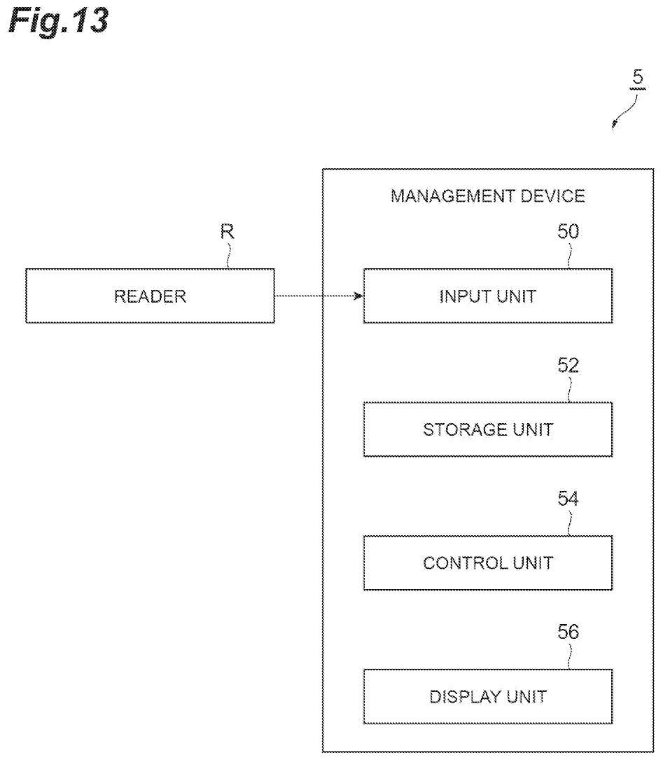

As illustrated m FIG. 13, the management device 5 has an input. unit 50, a storage unit 52, the control unit 54, and a display unit 56.

The input unit 50 inputs urinary output information measured by the measuring device 3. The input unit 50 inputs urinary output information received by the reader R from the measuring device 3. The reader R receives information transmitted from the measuring device 3 by short-distance wireless communication. The input unit 50 outputs input urinary output information to the storage unit 52.

The storage unit 52 is configured to have a ROM, a RAM, an FIDD, and the like. The storage unit 52 stores urinary output information input by the input unit 50 in association with patient information.

The control unit 54 is configured to have a CPU and the like.

For example, the control unit 54 controls display of the display unit 56 in accordance with an operation of an operation unit (not illustrated) such as a keyboard. For example, the display unit 56 is a display of a computer. For example, when an operation of requiring display of urinary output patient information is received, the control unit 54 acquires urinary output information from the storage unit 52 and causes the display unit 56 to display the acquired urinary output information,

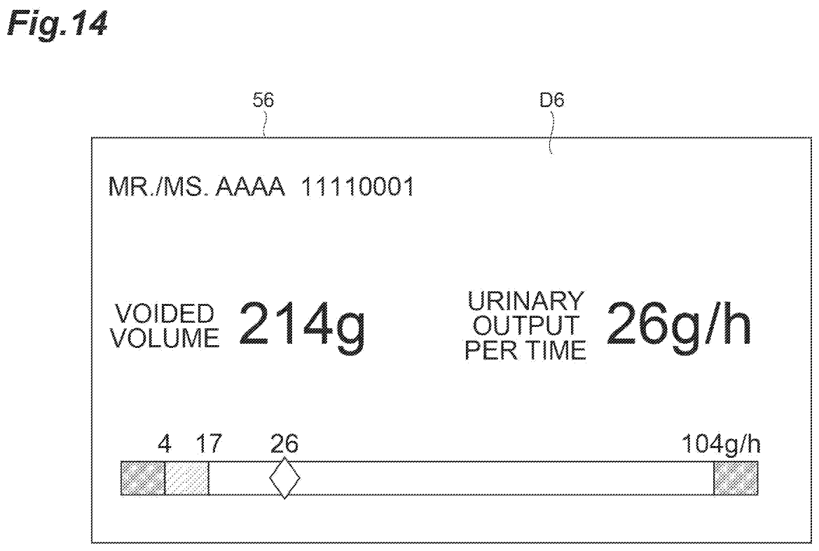

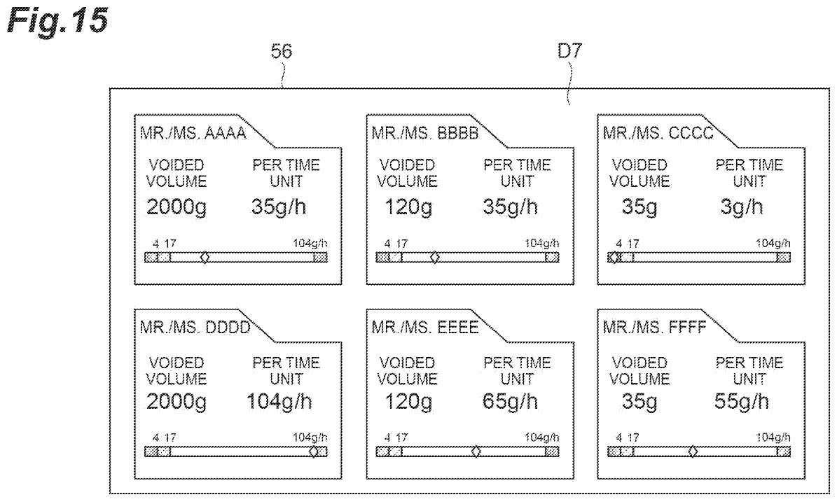

As illustrated in FIG. 14, for example, “voided volume” and “urinary output per time” are displayed for each patient in a display screen D6 of the display unit 56. The display screen D6 displays current urinary output per unit times for anuria, oliguria, and polyuria in a bar. In addition, as illustrated in FIG. 15, data of each patient may he displayed in a table in a display screen D7 of the display unit 56.

Subsequently, a method of utilizing the measuring system 1 will be described. In order to use the measuring device 3, first, the urine bag 100 is set to the measuring device 3. Subsequently, a power source button (not illustrated) of the measuring device 3 is pressed. Accordingly, the power source of the measuring device 3 is turned on. When the measurement button B is pressed, the measuring device 3 starts measurement. When the measurement button B is pressed, the measuring device 3 performs zero-point calibration. At this time, when equal to or more than a predetermined amount of urine is stored in the urine bag 100, that is, when urine of an amount equal to or more than “zero-point range” is stored in the urine bag 100, the measuring device 3 does not perform zero-point calibration, The measuring device 3 starts to measure a urinary output after zero-point calibration.

When a staff member acquires urinary output information, the measuring device 3 performs a predetermined operation, and the measuring device 3 transmits the urinary output information from the communication unit 18. The staff member brings the reader R of the management device 5 near the measuring device 3 and receives the urinary output information transmitted from the measuring device 3 by the reader R. When the urinary output information is received, the management device 5 inputs a value based on the urinary output information in electronic medical records.

As described above, in the measuring system 1 according to the present embodiment, the control unit 14 of the measuring device 3 causes the communication unit 18 to transmit urinary output information by short-distance wireless communication. In this configuration, urinary output information can be automatically acquired by the management device 5 by causing the management device 5 to receive the urinary output information transmitted from the communication unit 18 of the measuring device 3 by wireless communication. In this manner, in the measuring device 3, work of a staff member inputting a urinary output to electronic medical records for each patient can be omitted. Therefore, in the measuring device 3, it is I 0 possible to avoid occurrence of a human error when a. urinary output is registered in electronic medical records or a burden on a staff member when there is a need to cope with a plurality of patients. As a result, in. the measuring system 1 including the measuring device 3, work efficiency can be achieved.

In the measuring device 3, since not only urinary output information can be displayed in the touch panel display 16 but also urinary output information is output by wireless communication, a. staff member can acquire urinary output information without touching the urine bag 100. Therefore, a hygienic aspect when acquiring urinary output information can be ensured. Accordingly, at the time of rounds or the like to a hospital room by a staff member, the management device 5 can be carried into the hospital room, and urinary output information can be registered in electronic medical records. Therefore, since urinary output information can be registered in electronic medical records immediately after the urinary output information is acquired, work efficiency can be achieved.

In the measuring system 1 according to the present embodiment, the control unit 14 of the measuring device 3 transmits urinary output information from the communication unit 18 by short-distance wireless communication. In this configuration, urinary output information can be acquired in a non-contact manner by bringing the reader R provided in the management device 5 near the measuring device 3. Therefore, urinary output information (target) of the measuring device 3 can be reliably acquired.

In the measuring system 1 according to the present embodiment, the control unit 14 of the measuring device 3 acquires target person information indicating a target person using the urine bag 100 and manages the acquired target person information and urinary output information in association with each other. In this configuration, urinary output information can be managed for each target person (patient or the like).

In the measuring system 1 according to the present embodiment, the barcode reader BR reading a barcode indicating target person information can be connected to the measuring device 3. The control unit 14 acquires target person information on the basis of a barcode read by the barcode reader BR. In this configuration, target person information and urinary output information can be accurately associated with each other. Therefore, it is possible to reliably avoid occurrence of a problem such as occurrence of an error in association between target person information and urinary output information.

In the measuring system 1 according to the present embodiment, the control unit 14 of the measuring device 3 determines anuria, oliguria, and polyuria on the basis of urinary output information and a threshold set in advance and outputs notification information when it determines anuria, oliguria, or polyuria. In this configuration, a staff member can be notified of anuria, oliguria, or polyuria.

[Second Embodiment]

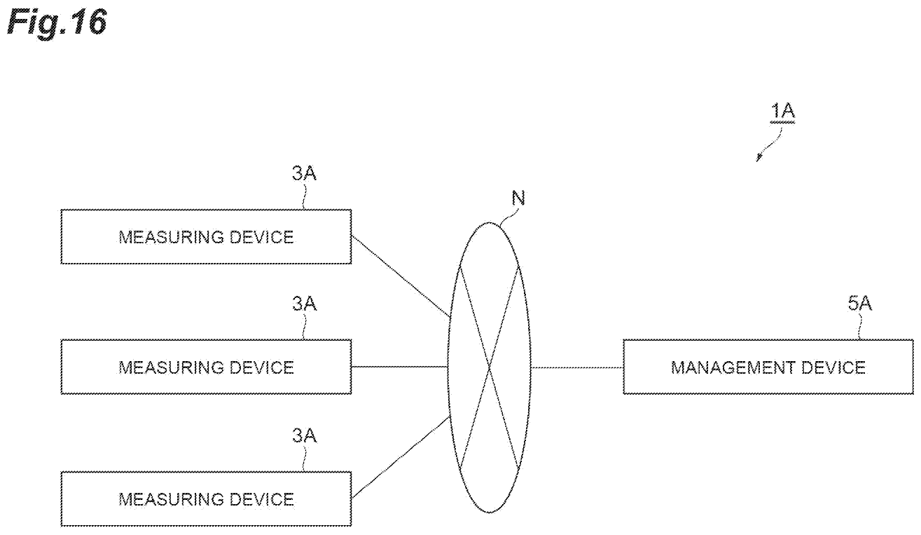

Subsequently, a second embodiment will be described. As illustrated in FIG. 16, a measuring system 1A according to the second embodiment includes measuring devices 3A and a management device 5A. In the present embodiment, a plurality of measuring devices 3A are provided. The measuring devices 3A and the management device 5A can communicate with each other via a network N such as a wireless local area network (LAN).

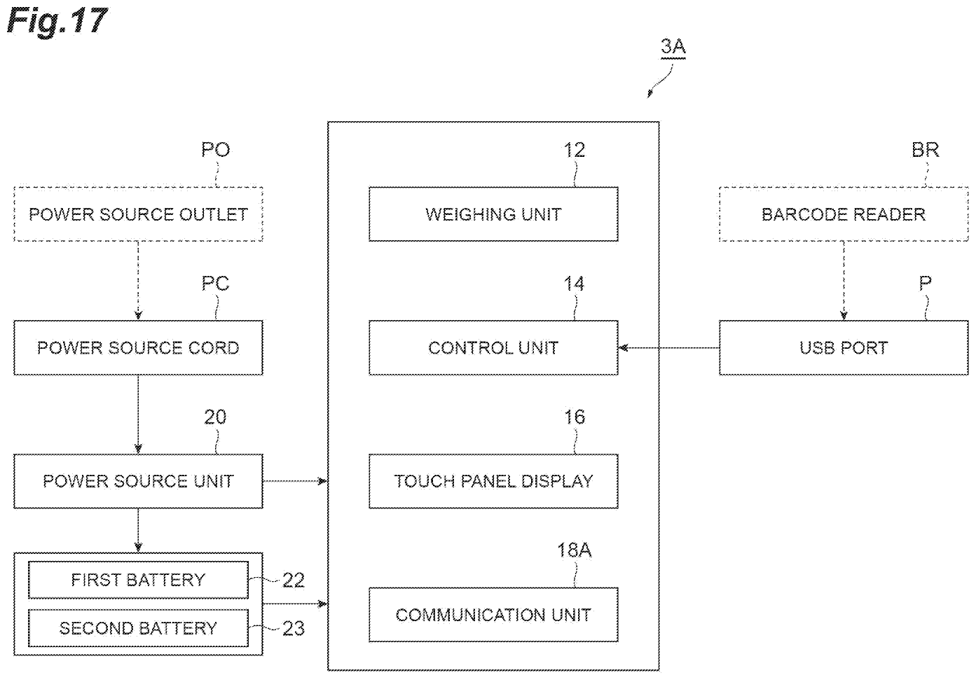

As illustrated in FIG. 17, each of the measuring devices 3A has the housing 10 (refer to FIGS. 2 and 3), the weighing unit 12, the control unit 14, the touch panel display 16, a communication unit 18A, the power source unit 20, the first battery 22, and the second battery 23.

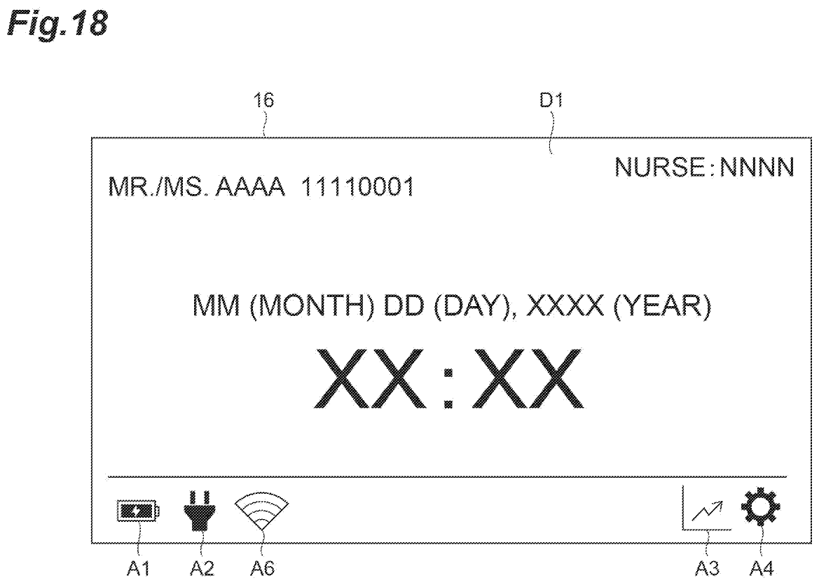

The control unit 14 causes the touch panel display 16 to display the standby screen D1 As illustrated in FIG. 18, the standby screen D1 displays “patient name”, “patient ID”, “nurse name”, “date and time”, “state icon”, “selection icon”, and the like. Regarding the “state icon”, for example, the residual quantity icon A1 indicating the state (residual quantity) of the first battery 22, the icon A2 indicating the connection state of the power source cord PC, and an icon A6 indicating the connection state (reception intensity) of the wireless LAN are displayed. Regarding the “selection icon”, the history icon A3 and the setting icon A4 are displayed.

When the setting icon A4 is touched in the standby screen D1, the control unit 14 causes the touch panel display 16 to display the setting screen. For example, items regarding wireless connection can he set in the setting screen. For example, regarding setting items for wireless connection, the setting screen displays “ON/OFF of Wi-Fi”, “Wi-Fi SSID”, “Wi-Fi password”, “security authentication method”, “IP address”, “subnet mask”, “IP address of gateway”, “MAC address”, “2.4 GHz/5 GHz”, and the like. In the setting screen, communication according to any of the standards of IEEE802.11b/g/n/a (2.4 GHz/5 GHz) can be set.

The control unit 14 determines anuria, oliguria, or polyuria on the basis of a urinary output per unit time. The control unit 14 determines anuria, oliguria, or polyuria on the basis of parameters set regarding anuria, oliguria, and polyuria set in advance. When the control unit 14 determines anuria, oliguria, or polyuria, the control unit 14 outputs notification information for causing the touch panel display 16 to display anuria, oliguria, or polyuria to the touch panel display 16 and outputs the notification information to the communication unit 18A.

The communication unit 18A is a wireless LAN module for wirelessly transmitting urinary output information. The communication unit 18A wirelessly transmits urinary output information in association with a unique identification code of the measuring devices 3A. Regarding a communication method of the communication unit 18A, various kinds of known wireless communication methods (IEEE802.11b/g/n/a) can be utilized. A timing when the communication unit 18A transmits urinary output information to the management device 5A may be a timing when urinary output information is generated in the control unit 14 or may be a predetermined time interval. When notification information is output. from the control unit 14, the communication unit 18A transmits notification information to the management device 5A.

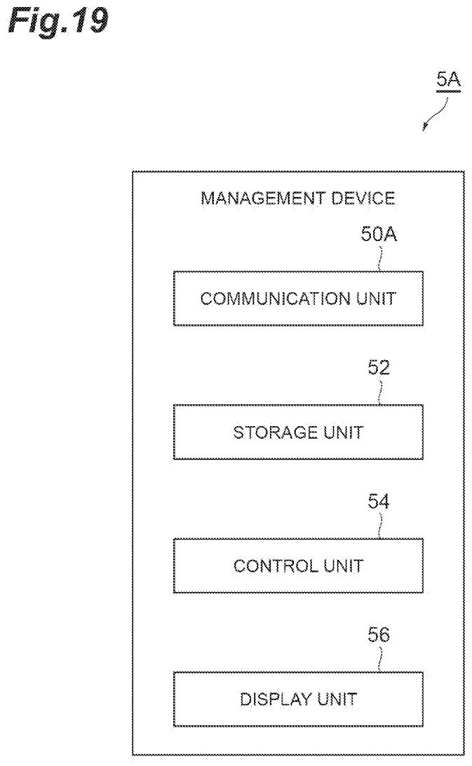

As illustrated in FIG. 19, the management device 5A has a communication unit 50A, the storage unit 52, the control unit 54, and the display unit 56.

The communication unit 50A is a wireless LAN module for wirelessly receiving urinary output information. The communication unit 50A outputs received urinary output information and an identification code to the storage unit 52. The storage unit 52 stores urinary output information received by the communication unit 50A in association with patient information.

The control unit 54 may receive an input of patient information and may transmit the patient information to the measuring devices 3A. In this configuration, the measuring devices 3A may receive patient. information transmitted from the management device 5A using the communication unit 18A and may register the patient information. In addition, when patient information read by the barcode reader BR coincides with patient information transmitted from the management device 5A, the measuring devices 3A may register the patient information.

As described above, in the measuring system 1A, the control unit 14 of the measuring devices 3A transmits urinary output information by wireless communication via a wireless LAN. In this configuration, urinary output information can be automatically acquired using the management device 5A by receiving the urinary output information transmitted by the communication unit 18A of the measuring devices 3A by wireless communication using the management device 5A. In this manner, in the measuring devices 3A, work of a staff member inputting a urinary output to electronic medical records for each patient can be omitted. Therefore, in the measuring devices 3A, it is possible to avoid occurrence of a human error when a urinary output is registered in electronic medical records or a burden on a staff member when there is a need to cope with a plurality of patients. As a result, in the measuring system 1A including the measuring devices 3A, work efficiency can be achieved.

In the measuring system 1A, even if a staff member does not visit a hospital room, urinary output information can be acquired in the management device 5A. Therefore, there is no need for a staff member to visit a hospital room and perform work of acquiring urinary output information. For this reason, in the measuring system 1A, reduction of a burden on a staff member can be achieved.

Hereinabove, embodiments of the present invention have been described. However, the present invention is not necessarily limited to the embodiments described above, and various changes can be made within a range not departing from the gist thereof.

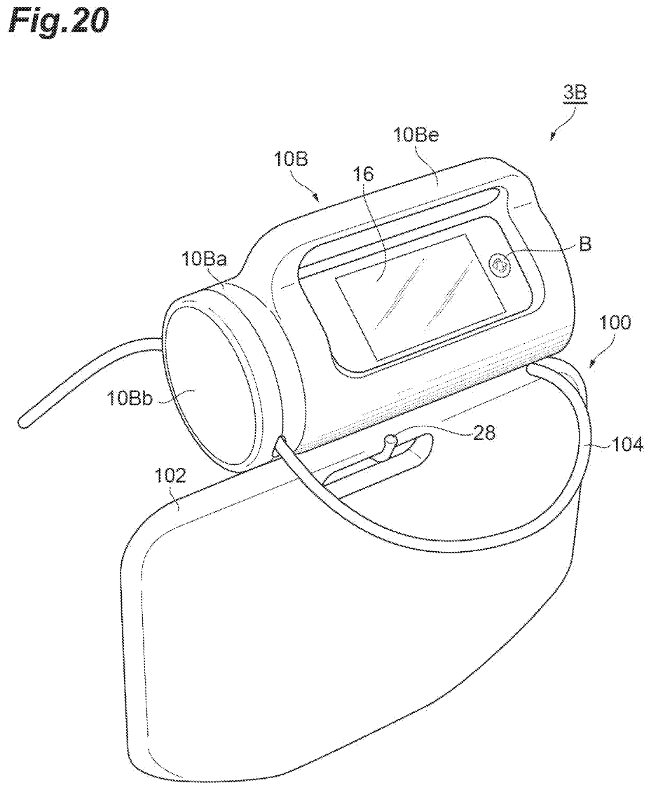

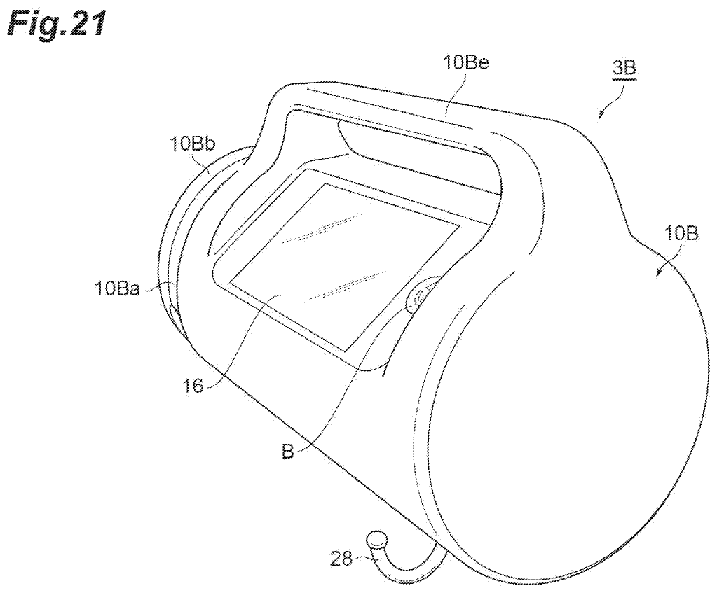

In the foregoing embodiments, a form in which the housing 10 of the measuring device 3 (3A) has a configuration (shape) illustrated in FIG. 1 has been described as an example. However, the configuration of a housing is not limited thereto. As illustrated in FIGS. 20, 21, and 22, a measuring device 3B has a housing 10B and the touch panel display 16. Similar to the measuring device 3 (3A), the measuring device 3B further has the weighing unit 12, the control unit 14, the communication unit 18 (18A), the power source unit 20, the first battery 22, and the second battery 23.

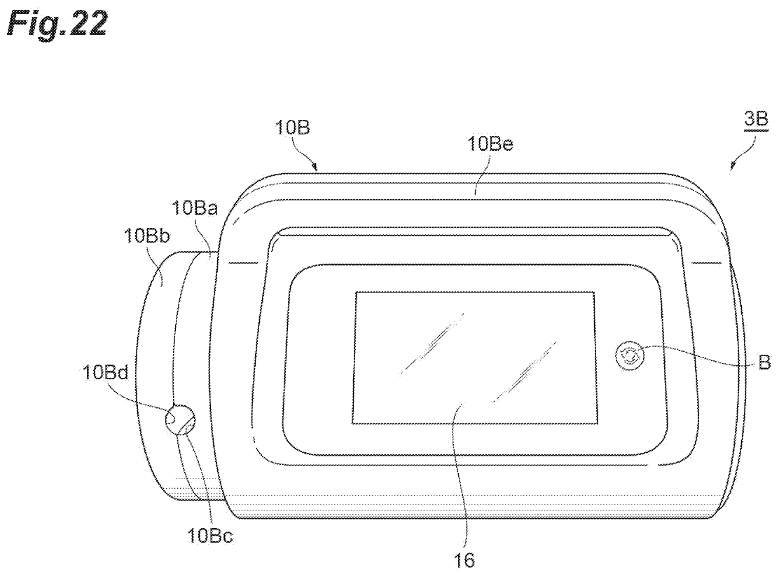

For example, the housing 10B is formed of an ABS resin or the like. The housing 1013 exhibits a cylindrical shape. The housing 10B has a holding portion 10Ba and a fixing portion 10Bb. The holding portion 10Ba is provided with a groove 10Bc in which the urine guide I0 tube 104 is disposed (refer to FIG. 22). The fixing portion 10Bb is provided at a position facing the holding portion 10Ba. The fixing portion 10Bb is provided in a swingable manner in the holding portion 10a. The fixing portion 10Bb is provided with a groove 10Bd ire which the urine guide tube 104 is disposed (refer to FIG. 22). The urine guide tube 104 can be attached to the holding portion 10Ba and the urine guide tube 104 can be detached from the holding portion 10Ba by opening the fixing portion 10Bb. The urine guide tube 104 can be sandwiched between the groove 10c and the groove 10Bd and the urine guide tube 104 can be fixed to the holding portion 10Ba by closing the fixing portion 10Bb. The fixing portion 10Bb restricts movement of the urine guide tube 104 in the holding portion 1.0Ba. The housing 10B is provided with a grip 10Be.

In the foregoing embodiments, a form in which the holding portion 10a or 10Ba and the fixing portion 10b or 10Bb are provided in the housing 10 of the measuring device 3, 3A, or 3B has been described as an example. However, the holding portion 10a or 10Ba and the fixing portion 10b or 10Bb may not be provided in the housing 10.

In the foregoing embodiments, a form in which the barcode reader BR is connected to the USB port P provided in the housing 10 or 10B of the measuring device 3 and a barcode including patient information is read by the barcode reader BR has been described as an example. However, a method of acquiring patient information is not limited thereto. For example, when patient information is included in a two-dimensional code, a device capable of reading a two-dimensional code may be connected to the USB port P. When patient information is included in an IC card in a non-contact manner, a device capable of reading an IC card in a ion-contact manner may be connected to the USB port P.

In the foregoing embodiments, a form in which the display unit is the touch panel display 16 in the measuring device 3, 3A, or 3B has been described as an example. However, the measuring device 3, 3A, or 3B may include a display serving as a display unit, keys (buttons) serving as an operation unit, and the like.

In the foregoing embodiments, a form in which the measuring device 3, 3A, or 3B includes the first battery 22 and the second battery 23 has been described as an example. However, the measuring device 3, 3A, or 3B may not include the first battery 22 and the second battery 23. In the foregoing embodiments, a form in which the first battery 22 and the second battery 23 are lithium-ion batteries has been described as an example. However, the first battery 22 and the second battery 23 may be different secondary batteries such as lead storage batteries. In the present invention, a secondary battery includes no capacitor.

In the foregoing embodiments, a form in which the control unit 14 of the measuring device 3, 3A, or 3B calculates the voided volume and the urinary output per unit time in [g] has been described as an example. However, the control unit 14 may calculate the voided volume and the urinary output per unit time in [ml]. In addition, the measuring: device 3, 3A, or 3B may calculate at least one of the voided volume and the urinary output per unit time.

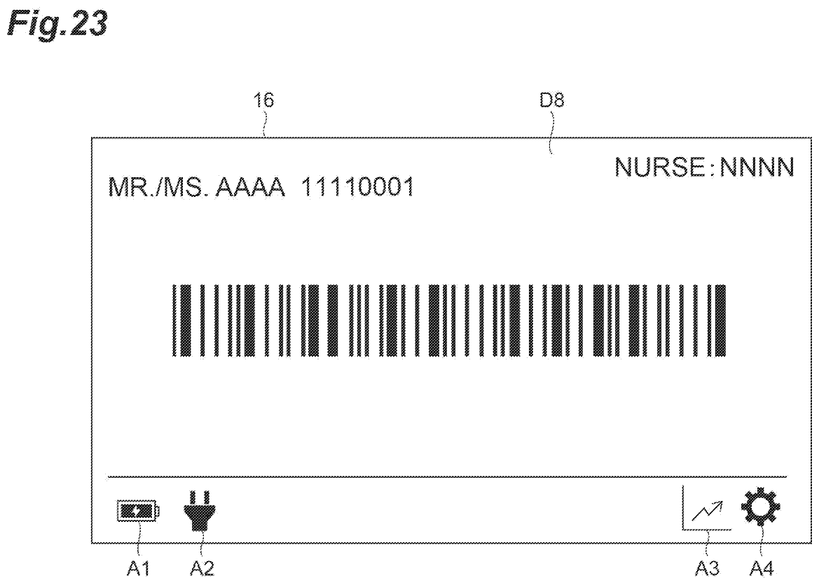

In the foregoing first embodiment, a form. in which the control unit 14 outputs urinary output information to the communication unit 18 when a predetermined operation is performed in the touch panel display 16 has been described as an example. However, when a. predetermined operation is performed in the touch panel display 16, the control unit 14 may generate a code including urinary output information and output the generated code to the touch panel display 16. As illustrated in FIG. 23, for example, the control unit 14 generates a barcode including urinary output information and causes the touch panel display 16 to display the barcode. The management device 5 acquires urinary output information by reading a barcode displayed in the touch panel display 16 of the measuring device 3 using the barcode reader.

In the measuring device 3, the control unit 14 may transmit urinary output information using the communication unit 18 or may cause the touch panel display 16 to display a barcode including urinary output information. In addition, the control unit 14 may switch between controls in accordance with an operation of a staff member.

Specifically, the touch panel display 16 displays a screen for selecting transmission of urinary output information or display of a barcode. The control unit 14 switches between methods for outputting urinary output information in accordance with an instruction received in the touch panel display 16.

In the foregoing first embodiment, each of the measuring device 3 and the management device 5 may he able to communicate with each other via a network such as a wireless LAN. In this configuration, similar to the measuring system 1A according to the second embodiment, urinary output information may be transmitted and received by communication via a wireless LAN. In this configuration, for example, in an area where Wi-Fi cannot be used, Wi-Fi may be turned off to directly acquire urinary output information from the measuring device 3 in the management device 5, and in an area where Wi-Fi can he used, Wi-Fi may he turned on such that the management device 5 acquires urinary output information from the measuring device 3 by wireless communication via a wireless LAN.

In addition to the foregoing embodiments, the measuring device 3, 3A, or 3B may acquire information regarding urine in addition to a urinary output. For example, the measuring device 3, 3A, or 3B may include a detection unit detecting blood included in urine, glucose included in urine, a specific weight of urine, urobilinogen included in urine, urinary protein, or the like. The detection unit may he provided in a manner of being attachable/detachable with respect to the housing 10 of the measuring device 3, 3A, or 3B. The detection unit is connected to the control unit 14 to he able to communicate with each other.

In the foregoing embodiments, a form in which the control unit 14 causes the touch panel display 16 to display the voided volume and the urinary output per unit time and urinary output information is output (transmitted) from the communication units 18 and 18A has been described as an example. However, in the measuring device, the output unit may he a printer or the like for printing urinary information or a code. In this case, the control unit 14 outputs urinary output information or information related to a barcode, for example, including urinary output information to the printer. The output unit prints urinary output information or a code on the basis of the information. The output unit may he provided in the housing 10 or 10B or may be provided separately from the housing 10 or 10B. Various forms may he employed as a printing method of the output unit.

### REFERENCE SIGNS LIST

1, 1A Measuring system

3, 3A, 3B Measuring device

5, 5A Management device

12 Weighing unit

14 Control unit (calculation unit, output unit, acquisition unit, management unit, determination unit)

16 Touch panel display (display unit)

18, 18A Communication unit

100 Urine hag

1. A measuring device acquiring information related to urine stored in a urine bag, the measuring device comprising:
a weighing unit configured to measure a mass value of the urine bag;
a calculation unit configured to calculate at least one of a voided volume and a urinary output per unit time on the basis of the mass value weighed by the weighing unit;
a display unit configured to display urinary output information related to at least one of the voided volume and the urinary output per unit time calculated by the calculation unit;
an output unit configured to generate a code including the urinary output information, output the code to the display unit, transmit the urinary output information by wireless communication, and/or print the urinary output information or the code; and
a control unit configured to switch an output destination in the output unit.

2. The measuring device according to claim 1,
wherein the output unit transmits the urinary output information by short-distance wireless communication.

3. The measuring device according to claim 1 further comprising:
an acquisition unit configured to acquire target person information indicating a target person using the urine bag; and
a management unit configured to manage the target person information acquired by the acquisition unit and the urinary output information in association with each other.

4. The measuring device according to claim 3,
wherein a reading device reading a code indicating the target person information is able to be connected to the measuring device, and
wherein the acquisition unit acquires the target person information on the basis of the code read by the reading device.

5. The measuring device according to claim 1 further comprising:
a determination unit configured to determine anuria, oliguria, and polyuria on the basis of the urinary output information and a threshold set in advance,
wherein the output unit outputs notification information when the determination unit determines anuria, oliguria, or polyuria.

6. A measuring system comprising:
the measuring device according to claim 1; and
a management device configured to acquire information related to urine acquired by the measuring device.
